(19) United States
(12) Patent Application Publication
(10) Pub. No.: US 2013/0217094 A1
(43) Pub. Date: Aug. 22, 2013

(54) ENZYME SOLUTION FOR SEPARATING CELL, METHOD FOR SEPARATING CELL AND METHOD FOR SEPARATING PANCREATIC ISLET

(71) Applicants: Akihiro Hazama, Fukushima (JP); Masao Miyake, Fukushima (JP); Mitsukazu Gotoh, Fukushima (JP); Takayuki Anazawa, Fukushima (JP); Naoshi Shinozaki, Tokyo (JP)

(72) Inventors: Akihiro Hazama, Fukushima (JP); Masao Miyake, Fukushima (JP); Mitsukazu Gotoh, Fukushima (JP); Takayuki Anazawa, Fukushima (JP); Naoshi Shinozaki, Tokyo (JP)

(73) Assignee: BIO LINK, INCORPORATED, Tokyo (JP)

(21) Appl. No.: 13/812,987

(22) PCT Filed: Jul. 27, 2011
(86) PCT No.: PCT/JP2011/067030

§ 371 Date: May 3, 2013

(30) Foreign Application Priority Data
Jul. 30, 2010 (JP) 2010-171867

(51) Int. Cl. C12N 5/071

(52) U.S. Cl. CPC C12N 5/0676
USPC 435/219; 435/381

(57) ABSTRACT

An enzyme solution for separating cell(s) which can lessen damage in separating a single cell or a mass of cells from a tissue or an organ, a method for separating cell(s) using the enzyme solution, and a method for separating pancreatic islet(s) using the enzyme solution. The enzyme solution for separating cell(s) is an enzyme solution containing a chloride ion channel inhibitor or having a chloride ion concentration of 10 mM or lower. By an enzymatic treatment using the enzyme solution for separating cell(s), a single cell or a mass of cells can be separated from a tissue or an organ. To separate pancreatic islet(s) from the pancreas, the enzyme is injected through the pancreatic duct to digest the pancreas.

### TECHNICAL FIELD

The present invention relates to an enzyme solution for separating cell(s), which separates a single cell or a mass of cells from a tissue or an organ, as well as a method for separating cell(s) using the enzyme solution and a method for separating pancreatic islet(s) using the enzyme solution.

### BACKGROUND ART

Much attention has been paid to a technique called transplantation of pancreatic islet to patients with type 1 diabetes who are in a so-called insulin-dependent state in which the life is not maintained without administration of insulin, and the technique is being established as a clinical treatment method mainly in countries in Europe and North America.

The transplantation of pancreatic islet is a technique of transplanting a cell tissue by infusing pancreatic islet that is a mass of cells which plays a central role for adjusting the blood glucose level in a portal vein in a living body by procedure of drip infusion. The transplantation of pancreatic islet has little invasion to the sides who undergo the transplantation, and therefore it is thought to be a substantially the most ideal treatment method for patients with type 1 diabetes.

Success in trial of clinical transplantation of pancreatic islet was reported in 2000 in Alberta University in Edmonton, Canada (see Non-Patent Literature 1). Since the report, a large number of transplantations of pancreatic islet have been carried out mainly in countries in Europe and North America. The transplantations of pancreatic islet are carried out based on the so-called “Edmonton Protocol” established in Alberta University.
Non-Patent Literature 1: Shapiro A M, Lakey J R, Ryan E A, et al., N. Engl. J. Med., 2000; 343:230-238
Non-Patent Literature 2: Brandhorst H, Brandhorst D, Hering B J, Bretzel R G, Transplantation, 1999; 68:355-361
Non-Patent Literature 3: Matsumoto S, Rigley T H, Reems J A, Kuroda Y, Stevens R B, Am. J. Transplant., 2003; 3:53-63
Non-Patent Literature 4: Lu W T, Lakey J R, Juang J H, Hsu B R, Rajotte R V, Transplant Proc., 2002; 34:2700-2701
Non-Patent Literature 5: Arita S, Une S, Ohtsuka S, et al., Pancreas, 2001; 23:62-67
Non-Patent Literature 6: Avila J G, Tsujimura T, Oberholzer J, et al., Cell Transplant., 2003; 12:877-881
Non-Patent Literature 7: Avila J, Barbaro B, Gangemi A, et al., Am. J. Transplant., 2005; 5:2830-2837
Non-Patent Literature 8: Goto T, Tanioka Y, Sakai T, et al., Transplantation, 2007; 83:754-758
Non-Patent Literature 9: Ichii H, Wang X, Messinger S, et al., Am. J. Transplant., 2006; 6:2060-2068

### DISCLOSURE OF THE INVENTION

### Problems to be Solved by the Invention

A problem of the transplantation of pancreatic islet is how to lessen damage to pancreatic islet in separating pancreatic islet(s). The pancreas is an organ for secreting digestive fluid, and the exocrine gland cells occupying of the most part thereof include various digestive enzymes. Enzymatic treatment for separating pancreatic islet(s) gives damage also on the exocrine gland cells, and causes release of digestive enzymes that are present in the exocrine gland cells. The digestive enzyme that exits to the outside of the cells has an extremely high tissue-toxicity, and gives much damage to pancreatic islet. To solve this problem is to radically enhance the success rate of the transplantation of pancreatic islet.

Conventionally, as a method of lessening damage to pancreatic islet, a method of carrying out enzymatic treatment at low temperatures (see Non-Patent Literature 2), a method using a trypsin inhibitor (see Non-Patent Literatures 3 and 4), a method using prostaglandin analogue (see Non-Patent Literature 5), a method using glutamine (see Non-Patent Literatures 6 and 7), a method using preoxygenated perfluorocarbons (see Non-Patent Literature 8), a method using nicotinamide (see Non-Patent Literature 9), and the like, are reported.

However, any of the methods cannot be sufficiently satisfactory methods, and new approaches for lessening the damage to pancreatic islet have been desired.

Note here that such lessening of damage is a common problem generated not only when pancreatic islet(s) is separated but also when a single cell or a mass of cells is separated from a tissue or an organ by enzymatic treatment. Lessening of damage to cells is desired also in, for example, regenerative medicine to the liver, the nerve, the blood vessel, or the like, separation of a cancer cell from a tissue, separation of a stem cell from a tissue, separation of the oocyte form the ovary in reproductive medicine, or the like.

The present invention has been made from the viewpoint of such conventional situations, and has an object to provide an enzyme solution for separating cell(s) which can lessen damage in separating a single cell or a mass of cells from a tissue or an organ, a method for separating cell(s) using the enzyme solution, and a method for separating pancreatic islet(s) using the enzyme solution.

### Means for Solving the Problems

The present inventor has keenly investigated to solve the above-mentioned problem. As a result, the present inventors have found that the above-mentioned problem is dissolved by adding a chloride ion channel inhibitor to an enzyme solution for separating cell(s) which is used for separating a single cell or a mass of cells from a tissue or an organ, or reducing the ion concentration of chloride in the enzyme solution, and have completed the present invention. The present invention specifically includes the followings.

(1) An enzyme solution for separating cell(s), including a chloride ion channel inhibitor.

(2) The enzyme solution described in the above (1) in which a concentration of the chloride ion channel inhibitor is 0.05 to 1 mM.

(3) An enzyme solution for separating cell(s) in which a chloride ion concentration is 10 mM or lower.

(4) The enzyme solution described in the above (3) in which the chloride ion is substituted by a negative ion other than a halide ion.

(5) A method for separating cell(s), the method including a step of separating a single cell or a mass of cells from a tissue or an organ by using an enzyme solution described in any of the above-mentioned (1) to (4).

(6) A method for separating pancreatic islet(s), the method including a step of degrading the pancreas by infusing an enzyme solution described in any of the above-mentioned (1) to (4) via the pancreatic duct.

### Effects of the Invention

The present invention can provide an enzyme solution for separating cell(s) which can lessen damage in separating a single cell or a mass of cells from a tissue or an organ, as well as a method for separating cell(s) using the enzyme solution, and a method for separating pancreatic islet(s) using the enzyme solution. In particular, when the present invention is applied for the method for separating pancreatic islet(s), a yield of pancreatic islet(s) or viability of the pancreatic islet(s), or the like, can be improved.

### BRIEF DESCRIPTION OF THE DRAWINGS

FIG. 1 is a graph showing a yield of pancreatic islets separated from rats of a control group, a DIDS group, and a low Cl⁻ group.

FIG. 2 is a graph showing viability of pancreatic islets separated from the rats of the control group, the DIDS group, and the low Cl⁻ group.

FIG. 3A is a graph showing TUNEL staining result of pancreatic islets separated from the rats of the control group.

FIG. 3B is a graph showing TUNEL staining result of pancreatic islets separated from the rats of the DIDS group.

FIG. 3C is a graph showing TUNEL staining result of pancreatic islets separated from the rats of the low Cl⁻ group.

FIG. 4A is a graph showing HE staining result of pancreatic islets separated from the rats of the control group.

FIG. 4B is a graph showing HE staining result of pancreatic islets separated from the rats of the DIDS group.

FIG. 4C is a graph showing HE staining result of pancreatic islets separated from the rats of the low Cl⁻ group.

FIG. 5A is a graph showing a change of a blood glucose level when the pancreatic islets separated from the rats of the control group were transplanted in the left kidneys of each of diabetes model SCID mice, and the pancreatic islet-transplanted kidney was extracted on Day 30 after transplantation.

FIG. 5B is a graph showing a change of a blood glucose level when the pancreatic islets separated from the rats of the DIDS group were transplanted in the left kidneys of each of diabetes model SCID mice, and the pancreatic islet-transplanted kidney was extracted on Day 30 after transplantation.

FIG. 5C is a graph showing a change of a blood glucose level when the pancreatic islets separated from the rats of the low Cl⁻ group were transplanted in the left kidneys of each of diabetes model SCID mice, and the pancreatic islet-transplanted kidney was extracted on Day 30 after transplantation.

FIG. 6 is a graph showing a change of a blood glucose level when an intraperitoneal glucose tolerance test was carried out with respect to diabetes model SCID mice on Day 25 after transplantation of pancreatic islet shown in FIGS. 5A-5C in comparison to a normal group of SCID mice in which the pancreatic islet is not transplanted.

### PREFERRED MODE FOR CARRYING OUT THE INVENTION

### Enzyme Solution for Separating Cell(s)

An enzyme solution in accordance with a first aspect of the present invention is characterized by containing a chloride ion channel inhibitor.

The chloride ion channel inhibitor is not particularly limited. Specific examples thereof include 4,4′-di(isothiocyano)stilbene-2,2′-disodium disulfonate (DIDS), 4-acetylamino-4′-isothiocyanato stilbene-2,2′-disodium disulfonate (SITS), 2-[(3-phenyl propyl)amino]-5-nitrobenzoic acid (NPPB), arachidonic acid, phloretin, 9-anthracenecarboxylic acid (9-AC), and the like.

The concentration of the chloride ion channel inhibitor is preferably 0.05 to 1 mM, and more preferably 0.1 to 0.5 mM.

An enzyme solution in accordance with the first aspect is similar to that of a conventional enzyme solution for separating cell(s) except that it contains a chloride ion channel inhibitor. That is to say, a solution in which an enzyme is added into a balanced salt solution such as a Hanks' solution can be used. As the enzyme, collagenase is generally used, but protease, dispase, or the like, may be used. The concentration of the enzyme is generally 0.5 to 2 mg/mL.

An enzyme solution in accordance with a second aspect of the present invention is characterized in that the chloride ion concentration is 10 mM or lower. The enzyme solution is the same as a conventional enzyme solution for separating cell(s) except that the chloride ion concentration is 10 mM or lower.

In order to make the chloride ion concentration be 10 mM or lower, a salt containing the other negative ions are only required to be used as negative ions instead of the salt containing chloride ions in preparation of an enzyme solution having a conventional composition. Examples of the other negative ions include a gluconic acid ion, a glucuronic acid ion, a glutamic acid ion, a sulfonic acid ion, a sulfuric acid ion, a boric acid ion, a nitrate ion, a carbonic acid ion, a bicarbonate ion, a phosphoric ion, an ascorbic acid ion, an oxalic acid ion, a citric acid ion, or the like. However, halide ions such as iodide ion and a bromide ion are not preferable because they have cytotoxicity. It is preferable that a positive ion is the same before and after substitution.

For example, it is possible to reduce the chloride ion concentration by substituting the sodium chloride in the composition of the enzyme solution with sodium glutamate.

Note here that the enzyme solution in accordance with the second aspect may contain a chloride ion channel inhibitor.

### Method for Separating Cell(s) and Method for Separating Pancreatic Islet(s)

A method for separating cell(s) in accordance with the present invention includes a step of separating a single cell or a mass of cells from a tissue or an organ, in particular, from an extracted tissue or organ, by using the enzyme solution of the present invention. The method for separating cell(s) can be widely applied to a region in which a single cell or a mass of cells is separated from a tissue or an organ by using an enzyme solution conventionally. Examples of such application fields include separation of pancreatic islet(s) from the pancreas, regenerative medicine to the liver, the nerve, the blood vessel, or the like, separation of a cancer cell from a tissue, separation of a stem cell from a tissue, separation of the oocyte from the ovary in reproductive medicine, or the like. Hereinafter, a method for separating pancreatic islet(s) from the pancreas is described as one example.

A method for separating pancreatic islet(s) includes a degradation step of degrading the pancreas by using an enzyme solution for separating cell(s), and a purification step of recovering pancreatic islet(s) from degraded pancreatic tissue.

### (Degradation Step)

In the case of animals including human, for degrading the pancreas, firstly, an enzyme solution of the present invention is infused via the pancreatic duct of the extracted pancreas so as to allow the pancreas to swell. Infusion of the enzyme solution can be carried out by infusing the enzyme solution into the main pancreatic duct by using, for example, a pump while an infusing pressure is adjusted.

After the enzyme solution is infused to the pancreas, the temperature of the enzyme solution is increased to about 37° C. by using an appropriate device so as to activate the enzyme. Thus, degradation can be started. When, for example, collagenase is used as the enzyme, the collagenase is activated according to the increase in the temperature, and by dissolving the collagen that forms a connective tissue, the pancreatic tissue is degraded.

Stopping of degradation can be carried out by lowering the temperature of the enzyme solution. Furthermore, the degradation can be stopped by adding serum protein (for example, albumin) so as to inactivate the enzyme.

After the degradation is stopped, pancreatic tissues are recovered. It is preferable that the recovered pancreatic tissue is concentrated by carrying out centrifugation before the purification step.

Furthermore, in the case of non-human animals, unlike the above, instead of infusing the enzyme solution after the pancreas is extracted, it is possible to infuse the enzyme solution before extraction. For example, the choledoch duct is clamped, and the enzyme solution is infused from the choledoch duct to the pancreas via the pancreatic duct. Thereafter, the pancreas is extracted, and incubated at a temperature of about 37° C., and thereby the degradation can be started. The method for stopping the degradation is similar to the above.

### (Purification Step)

Purification of pancreatic islets can be carried out by using the fact that the pancreatic islet has lighter specific gravity than that of pancreatic exocrine tissue. Specifically, by carrying out density gradient centrifugation using the recovered pancreatic tissue, the pancreatic islet(s) and the pancreatic exocrine tissue are separated from each other, and thereby the pancreatic islet(s) can be recovered.

### EXAMPLES

Hereinafter, the present invention is described in more detail with reference to Examples, but the present invention is not limited to these Examples.

Note here that in the following Examples, rats at an age of 9 to 10 weeks were used as a donor of the pancreatic islets. Furthermore, as a recipient, SCID mice at an age of 9 to 10 weeks were used. Streptozocin (STZ; 250 mg/kg) was intraperitoneally administered to SCID mice under anesthesia of diethyl ether, and β cells were destroyed to induce diabetes. Mice whose casual blood glucose level exceeds 350 mg/dL for two continuous days were regarded as diabetes mice, and they were used as the recipients.

### Example 1

### Separation of Pancreatic Islets from Rats

The rats were grouped into three groups (a control group, a DIDS group, a low Cl⁻ group) each including 8 rats, then abdominal section was carried out to each rat under ether anesthesia, the choledoch duct was clamped in the vicinity of the entrance of the duodenum, and cannula was inserted. Then, 12 mL of Hanks' solution (NaCl: 8000 mg/mL, KCl: 400 mg/mL, MgSG₄: 48.8 mg/mL, MgCl₂: 46.8 mg/mL, CaCl₂: 140 mg/mL, KH₂PO₄: 60 mg/mL, Na HPO₄: 47.9 mg/mL and glucose: 1000 mg/mL) containing collagenase (2 mg/mL) was infused into the pancreas via the pancreatic duct from the cannula. However, to the DIDS group, DIDS (200 μM) as a chloride ion channel inhibitor was further added, and to the low Cl⁻ group, sodium chloride in the Hanks' solution was substituted with sodium glutamate.

The pancreas that had been swollen by infusion of the solution was extracted, and incubated at 37° C. for 32 minutes. Thereafter, degraded tissue was placed into 50 mL conical tube, and the Hanks' solution at 4° C. was added so as to stop the enzyme reaction. The tube was gently shaken, and subjected to centrifugation at 320G for 10 to 15 seconds. Next, the pellet was washed with the Hanks' solution for three times, a tissue suspension was allowed to pass through a mesh filter (hole diameter: 860 μm) to remove a large not-degraded tissue, and then subjected to density gradient centrifugation (density: 1.120 g/cm³, 1.090 g/cm³, and 1.050 g/cm³) using Ficoll (type 400; manufactured by Sigma Chemical) to recover pancreatic islet. The recovered pancreatic islets were cultured in the CO₂ incubator at 37° C. by using RPMI1640 medium containing 2% fetal bovine serum.

For the yield of the pancreatic islets, IEQ (islet equivalents; the number of pancreatic islets based on the diameter of 150 μm) as a unit is used. Pancreatic islets obtained from each rat by using diphenyl thiocarbazone was subjected to staining and observation under microscope. The pancreatic islets were classified into groups each having a diameter of 50 to 100 μm, 101 to 150 μm, 151 to 200 μm, 201 to 250 μm, 251 to 300 μm, 301 to 350 μm, 351 to 400 μm, and 400 μm or more, and values multiplied by a coefficient, that is, N×0.167, N×0.667, N×1.685, N×3.5, N×6.315, N×10.352, N×15.833, and N×22.75, in which the number of pancreatic islets in each group was represented by N, were obtained. Then, the total value of the values was defined as the yield of the pancreatic islets from each rat. Note here that the number of pancreatic islets whose diameter was less than 50 μm was not counted.

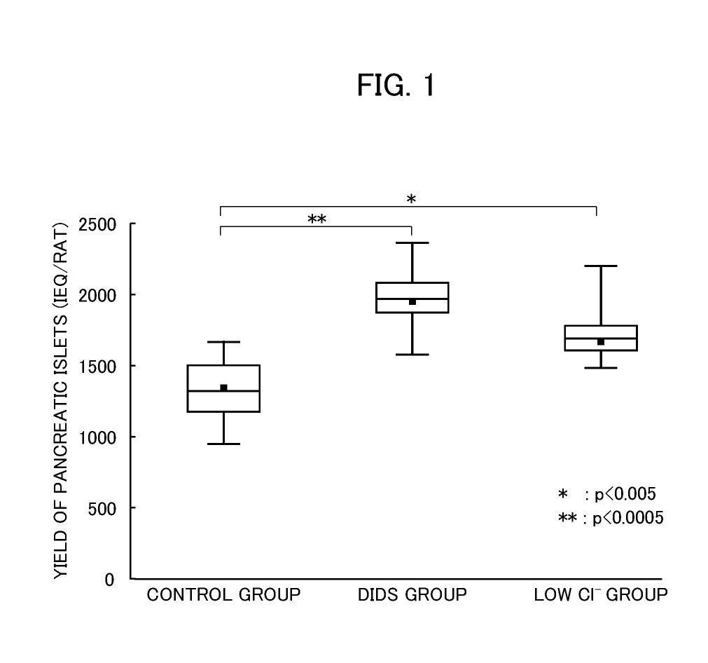

The yields of pancreatic islets separated from rats of the control group, the DIDS group and the low Cl⁻ group are shown in FIG. 1. As shown in FIG. 1, the yield of the pancreatic islets in the control group was 1316±245 IEQ. On the other hand, the yield of the pancreatic islets in the DIDS group was 1969±244 IEQ, and the yield of the pancreatic islets in the low Cl⁺ group was 1725±221 IEQ, which was significantly increased.

### Example 2

### Evaluation of Damages in Pancreatic Islets

The degree of damage of pancreatic islets can be evaluated based on whether or not a barrier function of the cell membrane can be maintained. Then, the pancreatic islets of each group separated in Example 1 (n=8 in each group) was stained with acridine orange (AO) and propidium iodide as fluorescent dye. Note here that AO is taken in cells even in living cells to emit green fluorescence, and PI is taken into cells by failure of the barrier function of the cell membrane and bound to DNA to emit red fluorescence. Specifically, AO (10 μM) and PI (15 μM) were dissolved in PBS to form a solution, the pancreatic islets were incubated in the resultant solution for 10 minutes, and then the pancreatic islets were observed under fluorescence microscope. Then, by using image analysis software (Image J free software), an area of the green fluorescence and an area of the red fluorescence were obtained, the rate (%) of the area of green fluorescence excluding the area of the red fluorescence was defined as the viability (%) of pancreatic islet.

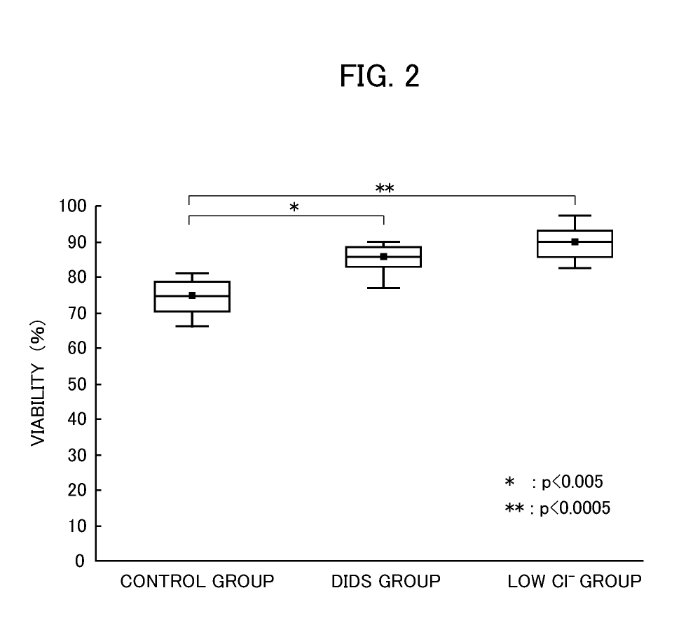

The viability of the pancreatic islets each separated from the rats of the control group, the DIDS group and the low Cl⁺ group is shown in FIG. 2. As shown in FIG. 2, the viability of the pancreatic islets in the control group was 74.6±5.6%. On the other hand, the viability of the pancreatic islets in the DIDS group was 84.9±4.9%, and the viability of the pancreatic islets in the low Cl⁻ group was 89.6±5.2%, which was significantly increased.

### Example 3

### Secretion of insulin by Glucose Stimulation

Abilities of secreting insulin with respect to stimulation with glucose having low concentration (3.3 mM) and glucose having high concentration (20 mM) in each group of pancreatic islets separated in Example 1 were measured. As the previous step of the measurement, pancreatic islets having a diameter of 150 to 200 μm (n=10 in each group) were cultured on a 12-pore Transwell microplate (Corning Transwell 3403; pore size of 12 μm) for 24 hours. As a medium, RPMI1640 medium containing 3.3 mM glucose and 0.1% fetal bovine serum was used, and conditions were set at 37° C. and in 5% CO₂/95% air condition.

In measurement, the Transwell medium on which pancreatic islets were placed was subjected to RPMI1640 medium containing 3.3 mM glucose and 0.1% fetal bovine serum, and pre-cultured for 60 minutes. Note here that the culture was carried out at 37° C. and in 5% CO₂/95% air conditions. Thereafter, the Transwell medium on which pancreatic islets were placed was moved to a new well containing the similar medium and stood still for 60 minutes (sample A). Furthermore, the Transwell medium on which pancreatic islets were placed was moved to a new well containing 20 mM glucose and stood still for 60 minutes (sample B). Thereafter, the media of the sample A and the sample B were recovered, and insulin contained in the medium was measured by using an insulin measurement kit (manufactured by Morinaga Biochemical Lab, Inc.) using the ELISA method. Then, the value obtained by dividing the insulin level of the sample B by the insulin level of the sample A was defined as an insulin secretion stimulation index. The results are shown in the following Table 1.

TABLE 1

| | insulin secretion (μU) | | |
|---|---|---|---|
| | sample A | sample B | insulin secretion |
| | (3.3 mM glucose) | (20 mM glucose) | stimulation index |
| control | 2.58 ± 0.97 | 18.7 ± 5.63 | 7.56 ± 1.82 |
| group | | | |
| DIDS | 1.94 ± 0.44 | 19.0 ± 4.46 | 9.93 ± 2.18^{a} |
| group | | | |
| low Cl⁻ | 1.73 ± 0.25^{a} | 19.4 ± 6.67 | 11.3 ± 3.90^{a} |
| group | | | |

^{a}p < 0.05 vs control group

As is apparent from Table 1, the insulin secretion stimulation index in the control group was 7.56±1.82. On the other hand, the insulin secretion stimulation index in the DIDS group was 9.93±2.18, and the insulin secretion stimulation index in the low Cl⁻ group was 11.3±3.90, which was significantly increased.

### Example 4

### TUNEL Staining and HE Staining

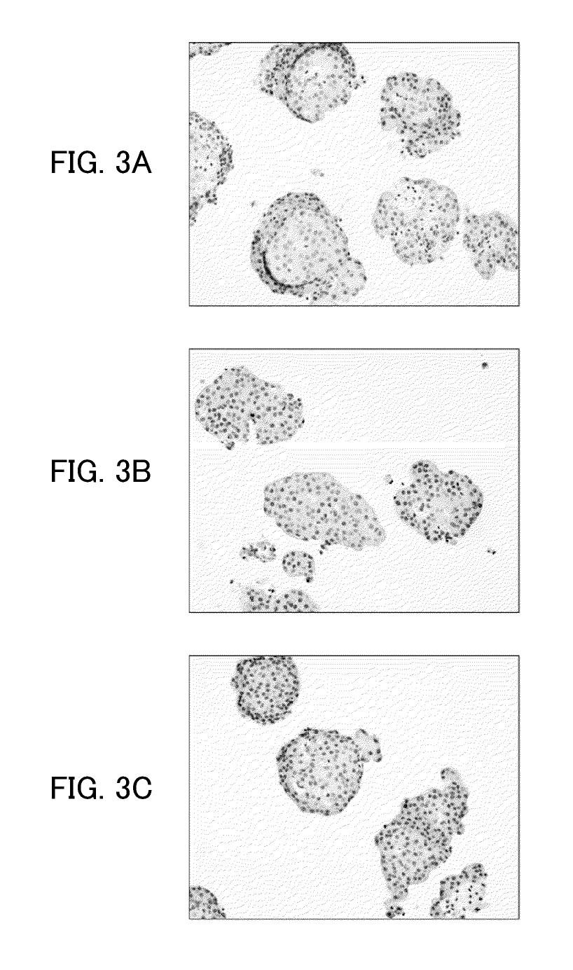

The pancreatic islet of each group separated in Example 1 was fixed by using 4% paraformaldehyde at 4° C. for one day, and subjected to paraffin embedding. Thereafter, 5 μm-thick section was formed and it was mounted on a preparation plate and subjected to TUNEL staining by using In Situ Apoptosis Detection Kit (manufactured by TAKARA BIO INC). The results of the TUNEL staining in the control group, the DIDS group, and the low Cl⁻ group are shown in FIGS. 3A, 3B, and 3C, respectively.

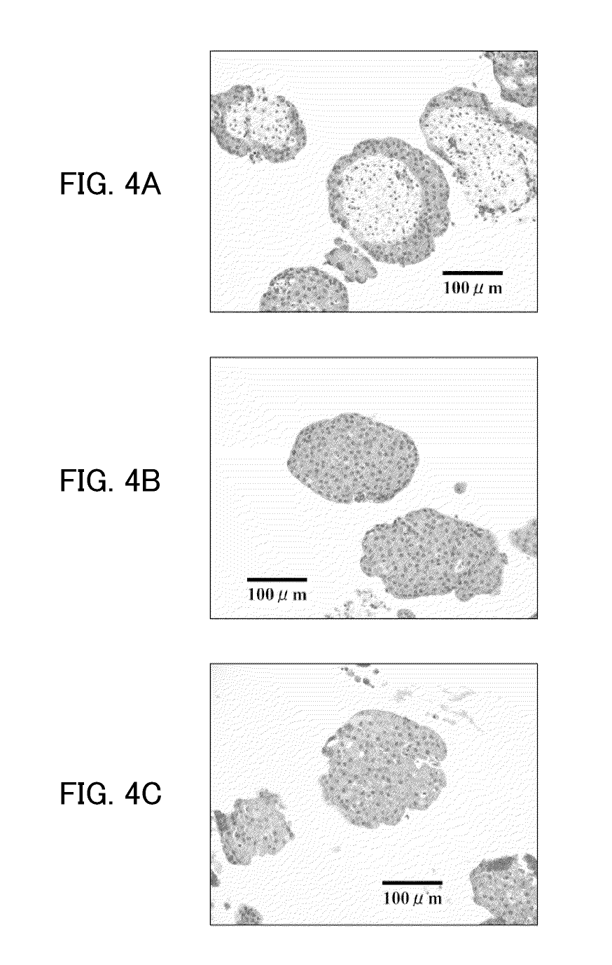

Furthermore, the pancreatic islets of each group separated in Example 1 were fixed by using 4% paraformaldehyde at 4° C. for one day, and subjected to paraffin embedding. Thereafter, 5 μm-thick section was formed and it was mounted on a preparation plate and subjected to HE staining. The results of the HE staining in the control group, the DIDS group, and the low Cl⁻ group were shown in FIGS. 4A, 4B, and 4C, respectively.

As is apparent from FIGS. 3A-3C, TUNEL-positive cells were hardly detected in any of the groups. Furthermore, as is apparent from FIGS. 4A-4C, a portion that had not been stained by HE staining was observed in the control group.

Thus, a cell death occurring during separation of pancreatic islets is thought to be necrosis generated due to swelling of a cell. By using the enzyme solution for separating a cell according to the present invention, such a cell death can be suppressed.

### Example 5

### Transplantation of Pancreatic Islets to Diabetes Model SCID Mice

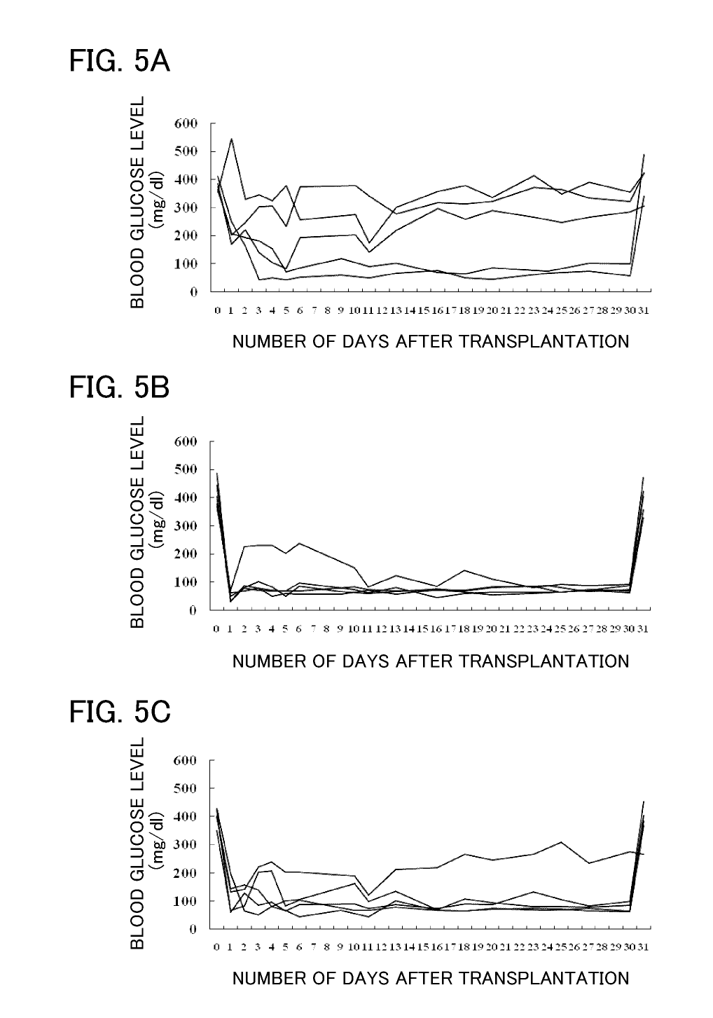

Pancreatic islets (200 IEQ) of each group separated in Example 1 was transplanted to the renicapsule of the diabetes model SCID mice (n=5 in each group) by STZ treatment. For the screening of the diabetes model SCID mice, mice whose casual blood glucose level exceeds 350 mg/dL for two continuous days by collection of blood were employed. In transplantation, the mice were subjected to ether anesthesia, and pancreatic islet was infused into the renicapsule of the left kidney by using a 250 μL pipette tip. After transplantation, the blood was recovered every day for the first seven days, and the blood glucose level was measured, and thereafter, the blood glucose level was measured three times a week. Then, on Day 30 after transplantation, the pancreatic islet-transplanted kidney was extracted. Note here that the definition of cure of diabetes was a case in which the blood glucose level was less than 150 mg/dL for three continuous days and the blood glucose level after nephrectomy was increased to more than 250 mg/dL. The measurement values of the blood glucose in the control group, the DIDS group and the low Cl⁻ group are shown in FIGS. 5A, 5B, and 5C, respectively.

As is apparent from FIGS. 5A-5C, the blood glucose level in the DIDS group on Day 1 to 2 after the transplantation (78.7±52.7 mg/dL) and the blood glucose level in the low Cl⁻ group (117.8±44.0 mg/dL) were significantly lower than the blood glucose level (252.3±107.4 mg/dL) in the control group (the DIDS group vs control group; p=0.0004, and the low Cl⁻ group vs control group; p=0.002). Furthermore, all of five mice in the DIDS group and four of five mice in the low Cl⁻ group came to have a normal blood glucose (<150 mg/dL), but only two of five mice in the control group came to have normal blood glucose.

All the mice which had normal blood glucose after transplantation were returned to hyperglycemia after the kidney was extracted.

### Example 6

### Intraperitoneal Glucose Tolerance Test (IPGTT) After Transplantation of Pancreatic Islets

In order to further examine the function of pancreatic islets, an intraperitoneal glucose tolerance test was carried out with respect to the mice on Day 25 after the transplantation of pancreatic islet in Example 5. The mice was subjected to 8-hour fasting before the test, and 2.5 g of glucose per kg of body weight was infused into the abdominal cavity of the mice in a state in which the glucose was added into a physiological saline solution. For mice, the diabetes model SCID mice which underwent transplantation of pancreatic islets (control group, the DIDS group and the low Cl⁻ group; n=5 in each group) and SCID mice which did not undergo transplantation of pancreatic islets (normal group; n=5) were used.

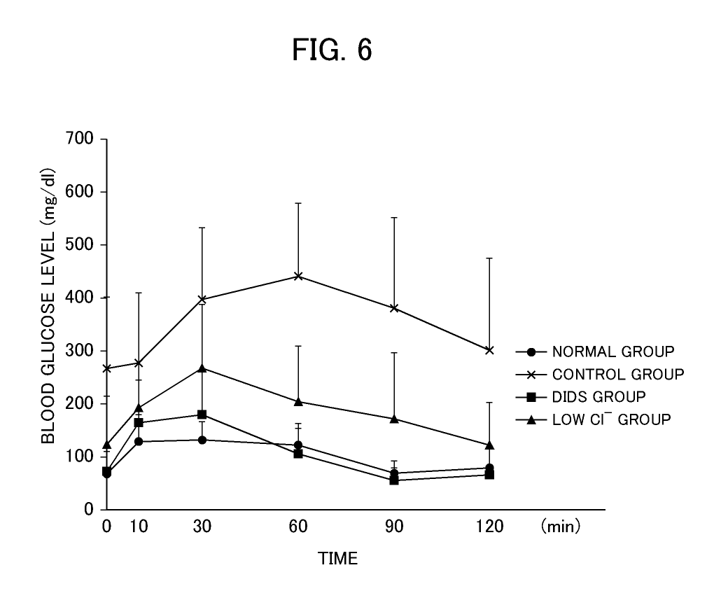

Then, the blood glucose level was measured before administration of glucose, and at the time when 10, 30, 60, 90, and 120 minutes had passed after the administration of glucose. The results are shown in FIG. 6.

As is apparent from FIG. 6, the change in the blood glucose level that is similar to the normal group was shown in the DIDS group and the low Cl⁻ group, but the control group was shifted in a state of the hyperglycemia.

1. An enzyme solution for separating cell(s), comprising a chloride ion channel inhibitor.

2. The enzyme solution according to claim 1, wherein a concentration of the chloride ion channel inhibitor is 0.05 to 1 mM.

3. An enzyme solution for separating cell(s) in which a chloride ion concentration is 10 mM or lower.

4. The enzyme solution according to claim 3, wherein the chloride ion is substituted with a negative ion other than a halide ion.

5. A method for separating cell(s), comprising a separating a single cell or a mass of cells from a tissue or an organ using the enzyme solution according to claim 1.

6. A method for separating pancreatic islet(s), comprising degrading the pancreas by infusing the enzyme solution according claim 1 via the pancreatic duct.

7. A method for separating cell(s), comprising separating a single cell or a mass of cells from a tissue or an organ using the enzyme solution according to claim 3.

8. A method for separating pancreatic islet(s), comprising degrading the pancreas by infusing the enzyme solution according to claim 3 via the pancreatic duct.
